(19) United States
(12) Patent Application Publication
(10) Pub. No.: US 2004/0121021 A1
(43) Pub. Date: Jun. 24, 2004

(54) Determination of adequate ozone disinfection

(76) Inventors: Zoran Minevski, The Woodlands, TX (US); Jason Maxey, Spring, TX (US)

Correspondence Address: STREETS & STEELE, 13831 NORTHWEST FREEWAY, SUITE 355, HOUSTON, TX, 77040, US

(21) Appl. No.: 10/325,663

(22) Filed: Dec. 19, 2002

(51) Int. Cl. C12Q 1/22; A61K 33/00

(52) U.S. Cl. 424/600; 435/31

(57) ABSTRACT

A substrate impregnated with an indicator that bleaches at an approximately linear rate in relation to the concentration of ozone in an aqueous solution. The color-impregnated substrate provides a visual determination that sufficient ozonation disinfection of a surface has occurred by using a direct measurement of the Ct value rather than just the measurement of an instantaneous concentration of ozone. The substrate may be used in a system for disinfecting a surface comprising means for contacting a surface to be disinfected with ozone, wherein one or more indicator-impregnated substrates are proximate to the surface; and means for comparing the second color to known colors of the indicator that correspond to different ozone Ct values. When the second color matches the known color for a target Ct value, a predetermined level of disinfection has been accomplished.

### BACKGROUND OF THE INVENTION

1. Field of the Invention

The present invention relates to an apparatus and method for determining the adequacy of disinfection. More particularly, the invention relates to a substrate that changes colors when contacted with ozone.

2. Description of the Related Art

According to recent reports to Congressional committees, many public health experts believe that the risk for food-borne illness is increasing. Over the last few years, numerous outbreaks have occurred including more than 100 cases of listeriosis from processed meats and at least 49 cases of E. coli from tainted lettuce. Food-borne pathogens may cause more than just discomfort, especially to the young, elderly or those with suppressed immune systems. Listeria monocytogenes has been known to cause stillbirths, miscarriages, meningitis and sepsis. E-coli 0157:H7 causes over 250 deaths in the United States per year. Cryptosporidium has become a major threat to the United States food and water supply, because it is resistant to chlorine, it is small and therefore difficult to filter, and it is ubiquitous in many animals.

Most produce and meat sold in United States markets have been washed with a disinfectant solution to remove or decrease the level of harmful pathogens. Ozone is an effective disinfectant for foodstuffs and is the preferred disinfectant for such pathogens since Cryptosporidium and other pathogens resistant to chlorine. The United States Environmental Protection Agency has developed a method for disinfection control that eliminates the need for direct monitoring for specific microorganisms to determine the adequacy of disinfection. Instead, the method requires monitoring and controlling the ozone contacting system to provide a predetermined level of disinfection. The method includes the concept of a “Ct value,” which is the mathematical product of the concentration of the disinfectant times the amount of time the surface to be disinfected is exposed to the disinfectant. This multiplication product captures the concept that microorganisms must be exposed to a sufficient dose of disinfectant over a sufficient period of time before the microorganism is destroyed. Therefore, a stronger disinfectant concentration may be applied over a shorter period of time to achieve a similar result as a weaker disinfectant concentration over a longer time period.

This method is based on the Chick-Watson Law, ln(Nₜ/N₀)=kCⁿt, describing the logarithmic inactivation of microorganisms. In this equation, Nₜ and N₀ are, respectively, the volumetric concentrations of microorganisms at time t and at the initial time, C is the residual concentration of a single disinfectant, n is assumed to be one, and t is time. When the disinfectant is ozone, the variable k is complex and is dependent on the microorganism that is being inactivated, the temperature, the pH and the water chemistry. Thus, different organisms, as well as varying water conditions, require different Ct values to achieve the same log reduction disinfection level.

In practice, the required Ct values are experimentally determined by plate culture counts for specific microorganisms and for specific disinfection processes to determine microorganism populations before and after exposure to ozone or other disinfectants. However, the results of these experimental determinations are limited to the conditions under which they were conducted. When ozone is the disinfectant, any change in the ozonation process, such as increased ozone demand in the wash water or slight changes in temperature or pH, may result in inadequate disinfection of the food. These changes may occur on a process-wide scale or may be limited to small areas in the process due to insufficient contacting of ozone with the wash water or poor distribution and/or flow patterns in the contactor. Therefore, proper determination of ozone disinfection is difficult using current methods that test only small areas for ozone concentration and may not account for large areas where disinfection may not be occurring.

Measuring ozone concentration in aqueous solutions is difficult due to the rapid decomposition of ozone, the volatility of ozone from solution, and the reactivity of the ozone with many organic and inorganic chemicals. It is important to note that most of the ozone analytical methods, which are modifications of the chlorine residual methods, are based on the determination of total oxidants in the solution and therefore, do not necessarily measure the ozone concentration alone.

Analytical methods for the determination of ozone concentration can be grouped into three distinct groups: physical, physicochemical and chemical methods. Physical methods are based upon measuring particular ozone properties, such as the intensity of absorption in the UV, visible or infrared region of the spectrum. The physicochemical methods measure physical effects of ozone with different reagents, such as chemiluminescence or heat of reaction. Chemical methods measure the quantity of reaction products that are released when ozone reacts with an appropriate reagent or the reduction in the molecular weight of a polymer.

These methods differ in sensitivity and accuracy and also share several common drawbacks. First, most of the methods detect and measure not only the ozone levels but also other oxidizing species. These methods may also experience interferences from ozonation by-products. Furthermore, most of the methods have a very limited detection range, usually less than 2 ppm. Finally, these methods are unsuitable for real-time on-line determination of ozone concentration, requiring independent sampling in order to measure the ozone concentration. Without real-time measurement systems, the determination of an accurate Ct value is prohibitive, requiring frequent sampling and testing of the disinfectant stream for ozone determination and fast procedures to reduce the turnaround time between sampling and Ct the determination of sufficient disinfection.

What is needed is a device that will accurately determine whether adequate disinfection has taken place. It would be an advantage if this device could be easily used and inexpensively produced. It would be a greater advantage if the device determined whether the target Ct value had been attained during a disinfection process.

### SUMMARY OF THE INVENTION

The present invention provides an apparatus for determining the adequacy of ozone disinfection comprising a substrate and at least one indicator or colorant. The substrate is impregnated by the indicator to a first color, which upon exposure to ozone over a period of time, changes to at least one different color. The different colors that the indicator changes to upon exposure to ozone over time each correspond to different ozone Ct values. The substrate may be of any porous material having an affinity for the indicator. For example, the substrate may be selected from the group consisting of nylon, polypropylene, polyethylene, vinyl, polytetrafluoroethylene, latex, cellulose, glass and combinations thereof. The substrate may be selected from paper, plastic or combinations thereof.

The at least one indicator is an organic substance, usually a dye or intermediate, that indicates by a change in its color the presence, absence, or concentration of ozone or the degree of reaction between ozone and contaminants. Optionally, the indicator may indicate a change in the concentration of ozone in combination with other oxidants. Preferably, the at least one indicator is selected from dye classifications selected from the group consisting of azo, indigoid, xanthene, anthraquinone, fluoran, quinoline, and combinations thereof. In one embodiment, the indicator is dissolved in a solvent, then impregnated into the substrate by a method of contact selected from the group consisting of dipping, spraying, painting and combinations thereof. The contact time between the indicator and the substrate is typically between about 1 second and about 30 minutes. Preferably, the contact time between the indicator and the substrate is between about 5 second and about 10 seconds. The contact temperature between the indicator and the substrate is typically between about 0° C. and 100° C. Preferably, the contact temperature between the indicator and the substrate is between about 25° C. and 40° C. Preferably, the indicator is dissolved in the solvent at a concentration from between about 1 ppm and about 10,000 ppm or more preferably from between about 500 ppm and about 2000 ppm.

The present invention further provides a method for determining an ozone Ct value, the method comprising placing one or more substrates proximate to a surface to be disinfected, wherein the substrate has been impregnated with an indicator having a first color; contacting the surface to be disinfected and the one or more substrates with ozone, wherein the ozone gradually oxidizes the indicator to a second color or shade; and comparing the second color to known colors that correspond to different ozone Ct values.

The ozone may be dissolved in water or the ozone may be gaseous, with or without other gases present as a mixture. The surface to be disinfected may be the surface of food, such as, for example, beef, pork, poultry, fish, fruits, vegetables, and combinations thereof. The ozone contacts the surface to be disinfected by a process selected from floating, spraying, dunking, immersing, mixing, washing and combinations thereof. For the disinfection process, the target Ct value is typically between about 1 ppm minute and about 300 ppm minutes. Preferably, the one or more substrates are placed proximate to the surface by a process selected from free-floating the substrate, attaching the substrate to the surface, attaching the substrate to a holder or combinations thereof.

The present invention further provides a system for disinfecting a surface, the system comprising means for contacting the surface with ozone, wherein one or more indicator-impregnated substrates are proximate to the surface and wherein the ozone gradually oxidizes the indicator to a second color or shade; and means for comparing the second color or shade to known colors of the indicator that correspond to different ozone Ct values. Attaining the target Ct value provides a predetermined level of disinfection. It should be recognized that the change in color or shade is preferably detectable by the human eye, but may instead be determined by a color detection device.

The foregoing and other objects, features and advantages of the invention will be apparent from the following more particular description of a preferred embodiment of the invention, as illustrated in the accompanying drawing wherein like reference numbers represent like parts of the invention.

### BRIEF DESCRIPTION OF THE DRAWINGS

FIG. 1 is a front view of examples of the substrate and the indicator changes.

FIG. 2 is a flowchart of a preferred method of disinfecting apples that may be implemented with the present invention.

FIG. 3 is a flowchart of a preferred method of disinfecting vegetables that may be implemented with the present invention.

FIGS. 4A-D are graphs showing the change in color intensity versus Ct value for different indicators impregnated in a substrate.

### DETAILED DESCRIPTION

The present invention provides a substrate impregnated with an indicator, preferably an indicator that bleaches at an approximately linear rate in relation to the concentration of ozone in a fluid. Therefore, the present invention provides a visual determination that a sufficient ozonation disinfection of a surface has occurred by using a direct measurement of the Ct value rather than just the measurement of an instantaneous concentration of ozone. Because the ozone concentration in a disinfection system may fluctuate widely throughout a process, focusing on spot ozone concentrations usually results in an unreliable measure of disinfection. The present invention allows the substrate to be exposed to the same ozone concentrations as the surface being disinfected, thereby providing a direct measurement of the Ct value. The present invention may be applied to any food disinfection process or other surface disinfection processes using ozone. Additionally, the present invention may be used in other industries using ozone, such as dry cleaning, chemical processes, or in products such as toothpastes or ointments that contain ozone.

The substrate may be any rough or porous material having an affinity for dyeing. The substrate may be, for example, paper, plastic or combinations thereof. Further examples include nylon, polypropylene, vinyl, polytetrafluoro-ethylene, latex, cellulose, glass or combinations thereof.

Preferred indicators for impregnating the substrate may be selected from dye or pigment classifications such as, for example, azo, indigoid, xanthene, anthraquinone, fluoran or quinoline. The dye may be soluble in any medium but is preferably insoluble in water.

The indicator may be applied to the substrate by a variety of methods. For example, the indicator may be dissolved in an appropriate solvent, such as acetone, and then applied to the substrate material by dipping, spraying or painting. According to this example, the concentration of the indicator in the solvent will typically range from 1 ppm to 10,000 ppm but is preferably in the range of 500 ppm to 2000 ppm. The time period necessary for contacting the indicator solution with the substrate material will typically vary from between about 1 second and about 30 minutes, but is preferably between about 5 seconds and about 10 seconds. The temperature of the indicator application will typically be between 0° C. and 100° C., but is preferably between about 25° C. and 40° C.

The choice of indicators and substrate may be made based upon the intended use of the colored substrate. Due to the differences in indicator activity and substrate material properties, such as porosity, wetability and surface roughness, a large range of ozone Ct values may be targeted. The range of Ct values targeted is preferably between about 1 ppm minute and about 300 ppm minutes.

Each kind of indicator-impregnated substrate, or Ct strip, must be tested to empirically determine the Ct value range most suitable for that particular combination of indicator and substrate. To empirically determine the Ct value range for a Ct strip having a particular indicator impregnated onto a particular substrate having particular properties, the particular Ct strip is exposed to known concentrations of ozone over incremental periods of time. The resulting color of the substrate is recorded after given increments of time until the color is entirely bleached out or until a maximum desired exposure time has passed. The color of the Ct strip, after each time increment of immersion in a known concentration of ozone, is an accurate measure of the Ct value for an exposed surface. The resulting colors are recorded for comparison use, as on a color chart, for that particular combination of indicator and substrate.

The preferred combination of indicator and substrate for a given target Ct value will be a combination that yields a fully bleached indicator or colorant at the target Ct value. In the preferred combination, when the Ct strip has no indicator left, then the Ct strip has been exposed for the target Ct value. However, this preferred combination might not always be available for a given target Ct value. Therefore, a Ct strip may alternatively be used that results in a color change, either a different color or a different shade of the first color, indicating that the target Ct value has been reached. By comparing the color of a Ct strip that has been exposed to ozone with the reference color chart for that type of Ct strip, the actual Ct time may be determined.

The present invention also provides a method for using the indicator-impregnated substrate to determine the adequacy of ozone disinfection. When a surface is disinfected using an ozonated process, the surface to be disinfected is intimately contacted with the gaseous or aqueous solution of ozone. The means for accomplishing the intimate contacting of a surface with ozone may be achieved, for example, by floating, spraying, dunking, immersing, mixing, washing or combinations thereof. For example, large vats filled with an ozone solution may be used for dunking, mixing or floating the items having surfaces to be disinfected. As another example, spray headers and nozzles may be used to spray an ozonated solution or ozone gas on the surface to be disinfected. Any means is acceptable that provides adequate disinfectant coverage of the surface to be disinfected.

The indicator-impregnated substrate is also placed in intimate contact with the gaseous or aqueous solution of ozone and proximate to the surface to be disinfected. One or more of the indicator-impregnated substrates may be used to ensure an adequate measurement of the Ct value during the disinfection process. If the process is, for example, in a well-mixed vat containing the ozonated solution and the surfaces to be disinfected, only one indicator-impregnated substrate may be necessary to measure the Ct value during the process because the process is well mixed. If, however, the process is a sprayed process over a relatively large area, then several indicator-impregnated substrates may be required. In this case, placing the substrates at several locations, especially at those locations that may receive the least amount of spray over the treatment period as, for example, at the corners or boundaries of the area being sprayed, will ensure that the indicator-impregnated substrates measure the Ct value for those areas requiring the longest contact time. It is important that a sufficient number of indicator-impregnated substrates be used during each disinfection procedure to provide an adequate measure of the Ct value for each of the areas of the surfaces to be disinfected. The indicator-impregnated substrates may be proximately located to the surfaces to be disinfected by placing them as free-floating substrates in a mixed vat, by attaching them directly to the surfaces to be disinfected, or by attaching them to free floating chemically inert holders that may be easily retrieved, such as by a cord, or magnetic attraction, or size exclusion, or by any other suitable means.

Because the indicator-impregnated substrates have been made to bleach at a substantially linear rate with respect to the Ct value, it is possible to visually compare the color of the substrate in the disinfectant process with a known chart of the indicator at a given Ct value. When the indicator in the disinfectant process has bleached to the color that is known to coincide with the target Ct value, then the disinfection process is complete and the surfaces may be considered to be disinfected.

The method of the present invention has several advantages over existing ozone detection processes. First, a wide range of Ct values can be measured in real-time by visual color change. This allows for immediate reaction to inadequate disinfection, reducing the potential for recalls of contaminated food products. Also, the method bleaches linearly as a function of Ct and not simply of ozone concentration, thus providing a reliable direct measure of disinfection rather than only localized ozone concentrations. The method also has a broad applicability to any ozone disinfection process, as the Ct strips are preferably designed or selected to be relatively insensitive to other oxidants commonly used in food disinfection.

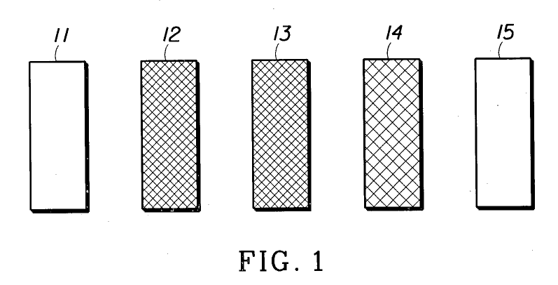

FIG. 1 is a front view of several substrates illustrating the indicator changes. A substrate with no indicator 11 appears white when the substrate is white. Preferably, the substrate is white to limit the interaction of the indicator with other colors. Alternatively, the substrate may be any other color that does not prohibit recognition of the indicator change during exposure to ozone. A substrate impregnated with an indicator forms a Ct strip 12 ready to be contacted with ozone. Immediately after being exposed to ozone, the Ct strip 13 will retain substantially the same color as the Ct strip originally had before exposure 12. After being exposed to the ozone for a set Ct value, the Ct strip 14 will become bleached out to a different known shade for that particular Ct strip. Finally, after a known Ct value for the particular substrate/indicator combination, the Ct strip 15 will be bleached out the original substrate color 11.

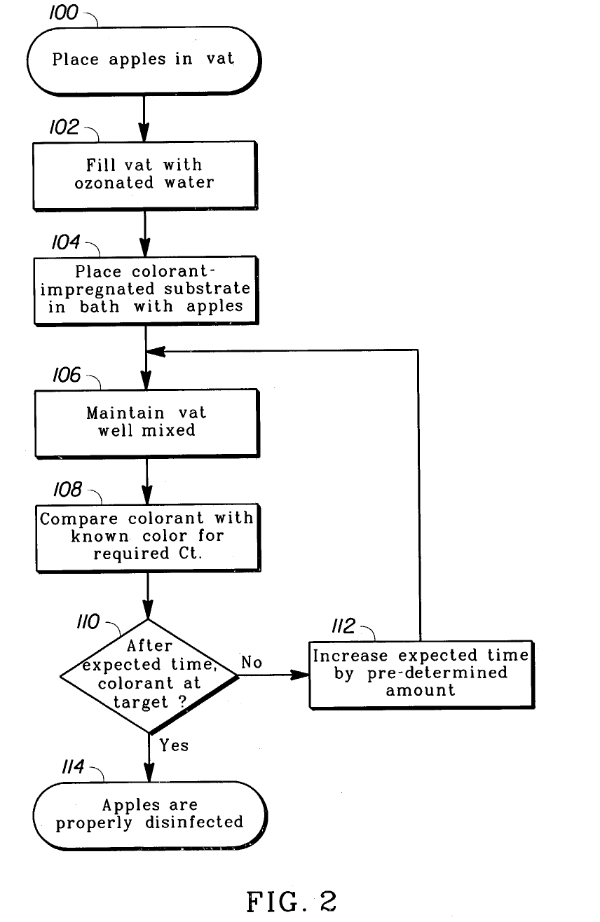

FIG. 2 is a flowchart of a preferred method of disinfecting apples that may be implemented with the present invention. In state 100, apples are placed in a vat for disinfection. In state 102, the vat is filled with ozonated water. In state 104, at least one indicator-impregnated substrate is placed in the vat secured to a floating holder that submerges the substrate. Preferably the size and weight characteristics of the holder are similar to those of the apples themselves so that the holder and apples obtain a similar experience within the vat. In state 106, the vat is mixed, either with a mixer in the vat or by circulating the ozonated water to ensure good mixing and a relatively uniform concentration of ozone throughout the vat. In state 108, the indicator is compared with the known color that the indicator will achieve when the Ct value target required for adequate disinfection has been achieved. If, in state 110, after the expected time for disinfection, the indicator has not reached the target color for adequate disinfection, then in state 112, the expected time period is increased by a predetermined amount and the method repeats states 106 through 110 as discussed above. If, in state 110, the indicator has reached the target color for adequate disinfection, then in state 114, the apples have been properly disinfected and may be packed and shipped to market.

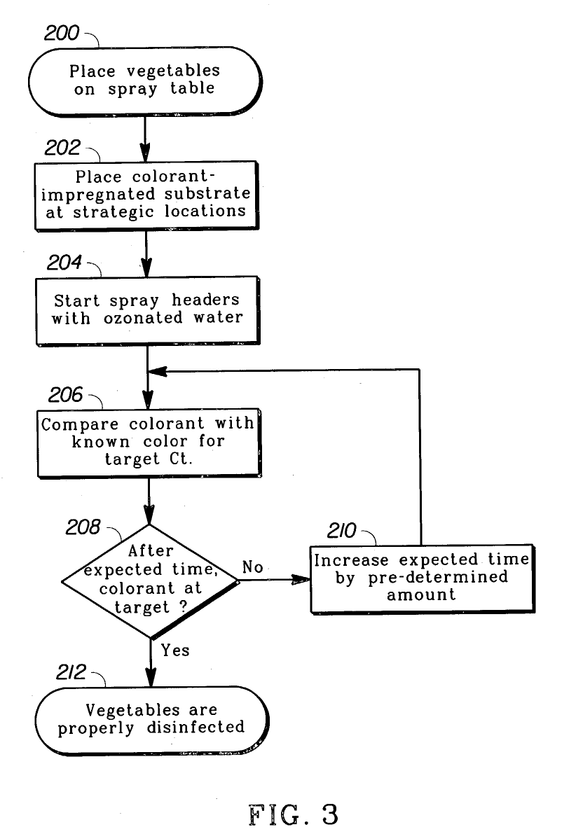

FIG. 3 is a flowchart of a preferred method of disinfecting vegetables that may be implemented with the present invention. In state 200, the vegetables are placed on a spray table. In state 202, the indicator-impregnated substrates are placed in strategic location around the perimeter of the table and on the table to provide a representative sampling of the disinfection process, especially in those areas that may not get as much disinfectant spray as other areas. In sate 204, the spray headers are started and ozonated water is sprayed on the vegetables. In state 206, the indicator is compared with the known color that the indicator will achieve when the Ct value target required for adequate disinfection has been achieved. If, in state 208, after the expected time for disinfection, the indicator has not reached the target color for adequate disinfection, then in state 210, the expected time period is increased by a predetermined amount and the method repeats states 206 and 208 as discussed above. If, in state 208, the indicator has reached the target color for adequate disinfection, then in state 212, the vegetables have been properly disinfected and may be packed and shipped to market.

### EXAMPLE 1

### Impregnation of Solvent Green 3 Dye on a Filter Paper Substrate and Exposure to Ozonated Water

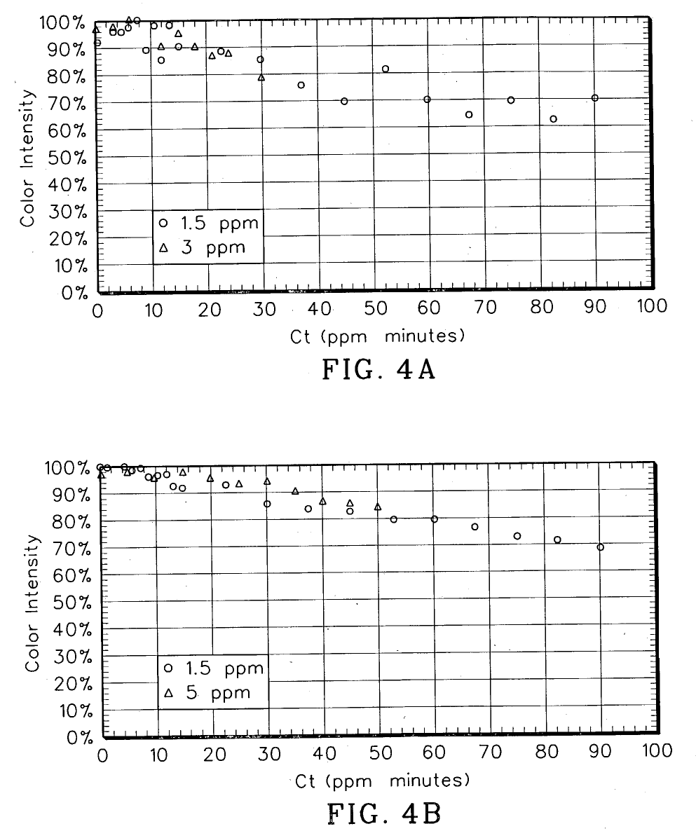

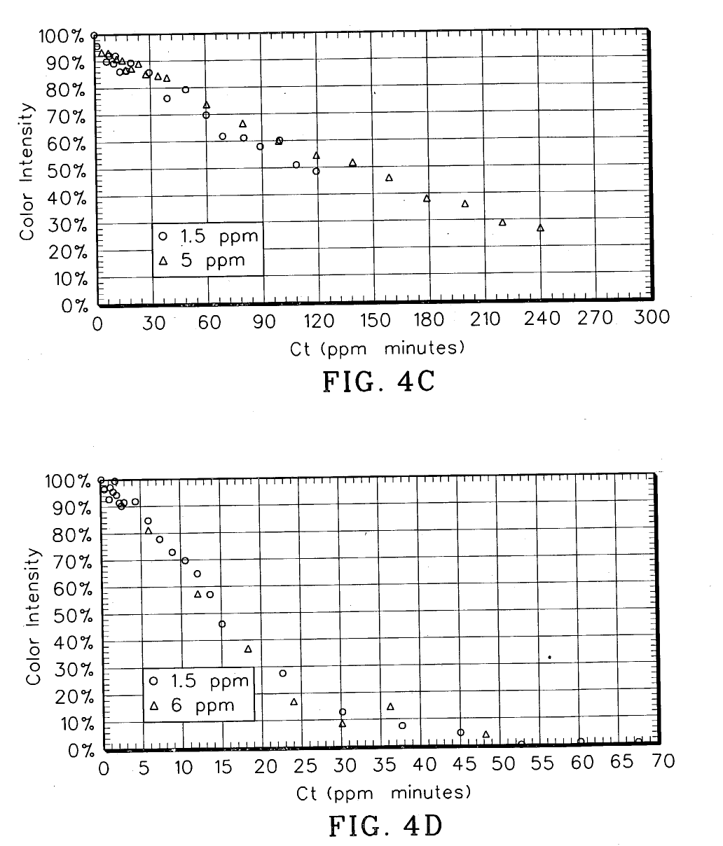

Strips of filter paper ½″×2″ were cut and immersed in a 1000 ppm solution of Solvent Green 3 (1,4-bis[(4-methylphenyl)amino]-9,10-anthracenedione) in acetone for 5 seconds at room temperature. When dyed in this manner, strips were initially an extremely dark green and dried to a medium green color. When submerged in a flowing bath of 3 ppm ozone dissolved in water, bleaching was observed. The color of the strip exhibited a linear decrease with time, resulting in approximately a 40% color loss after 90 minutes (see FIG. 4A).

### EXAMPLE 2

### Impregnation of Solvent Red 23 Dye on a Filter Paper Substrate and Exposure to Ozonated Water

Strips of filter paper ½″×2″ were cut and immersed in a 1000 ppm solution of Solvent Red 23 in acetone for 5 seconds at room temperature. When submerged in a flowing bath of 5 ppm ozone dissolved in water, bleaching was observed. The color of the strip exhibited a linear decrease with time, resulting in approximately a 30% color loss after 90 minutes (see FIG. 4B).

### EXAMPLE 3

### Impregnation of Solvent Violet 13 Dye on a Filter Paper Substrate and Exposure to Ozonated Water

Strips of filter paper ½″×2″ were cut and immersed in a 1000 ppm solution of Solvent Violet 13 in acetone for 5 seconds at room temperature. When submerged in a flowing bath of 5 ppm ozone dissolved in water, bleaching was observed. The color of the strip exhibited a linear decrease with time, resulting in approximately a 75% color loss after 240 minutes (see FIG. 4C).

### EXAMPLE 4

### Impregnation of Solvent Yellow 33 Dye on a Filter Paper Substrate and Exposure to Ozonated Water

Strips of filter paper ½″×2″ were cut and immersed in a 500 ppm solution of Solvent Yellow 33 in acetone for 5 seconds at room temperature. When submerged in a flowing bath of 6 ppm ozone dissolved in water, bleaching was observed. The color of the strip exhibited a linear decrease with time, resulting in complete color loss after 20 minutes (see FIG. 4D).

It will be understood from the foregoing description that various modifications and changes may be made in the preferred embodiment of the present invention without departing from its true spirit. It is intended that this description is for purposes of illustration only and should not be construed in a limiting sense. The scope of this invention should be limited only by the language of the following claims.

What is claimed is:

1. An apparatus for determining the adequacy of ozone disinfection comprising:
a substrate; and
an indicator, wherein the substrate is impregnated by the indicator to establish a first color, and wherein the indicator is capable of being gradually oxidized by ozone to at least one different color or shade, each of the at least one different colors or shades corresponding to a different ozone Ct value.

2. The apparatus of claim 1, wherein the substrate is selected from the group consisting of nylon, polypropylene, polyethylene, vinyl, polytetrafluoroethylene, latex, cellulose, glass and combinations thereof.

3. The apparatus of claim 1, wherein the substrate is selected from paper, plastic or combinations thereof.

4. The apparatus of claim 1, wherein the substrate is any porous material with a rough surface having an affinity for dyeing.

5. The apparatus of claim 1, wherein the indicator is selected from dye classifications selected from the group consisting of azo, indigoid, xanthene, anthraquinone, fluoran, quinoline, and combinations thereof.

6. The apparatus of claim 5, wherein the indicator is dissolved in a solvent before impregnating the substrate.

7. The apparatus of claim 6, wherein the substrate is impregnated with the dissolved indicator by a method of contact selected from the group consisting of dipping, spraying, painting and combinations thereof.

8. The apparatus of claim 7, wherein the contact time between the indicator and the substrate is between about 1 second and about 30 minutes.

9. The apparatus of claim 7, wherein the contact time between the indicator and the substrate is between about 5 second and about 10 seconds.

10. The apparatus of claim 7, wherein the contact temperature between the indicator and the substrate is between about 0° C. and 100° C.

11. The apparatus of claim 7, wherein the contact temperature between the indicator and the substrate is between about 25° C. and 40° C.

12. The apparatus of claim 5, wherein the concentration of indicator dissolved in the solvent is from between about 1 ppm and about 10,000 ppm.

13. The apparatus of claim 5, wherein the concentration of the indicator dissolved in the solvent is from between about 500 ppm and about 2000 ppm.

14. A method for determining an ozone Ct value comprising:
placing one or more substrates proximate to a surface to be disinfected, wherein the substrate is impregnated with an indicator to establish a first color;
contacting the surface to be disinfected and the one or more substrates with ozone, wherein the ozone gradually oxidizes the indicator to a second color or shade; and
comparing the second color or shade to known colors or shades that correspond to different ozone Ct values.

15. The method of claim 14, wherein the ozone is dissolved in water.

16. The method of claim 14, wherein the ozone is dissolved in an organic solvent.

17. The method of claim 14, wherein the ozone is gaseous.

18. The method of claim 14, wherein the surface is selected from the group consisting of beef, pork, poultry, fish, fruits, vegetables, and combinations thereof.

19. The method of claim 14, wherein the surface is the surface of food.

20. The method of claim 14, wherein the step of contacting the surface is accomplished by a process selected from the group consisting of floating, spraying, dunking, immersing, mixing, washing and combinations thereof.

21. The method of claim 14, wherein a target Ct value is between about 1 ppm minute and about 300 ppm minutes.

22. The method of claim 14, wherein the step of placing one or more substrates proximate to the surface is accomplished by a process selected from free-floating the substrate, attaching the substrate to the surface, attaching the substrate to a holder or combinations thereof.

23. The method of claim 14, wherein the one or more substrates is selected from the group consisting of nylon, polypropylene, vinyl, polytetrafluoroethylene, latex, cellulose, glass and combinations thereof.

24. The method of claim 14, wherein the one or more substrates is any porous material with a rough surface having an affinity for dyeing.

25. The method of claim 14, wherein the one or more substrates is selected from paper, plastic or combinations thereof.

26. The method of claim 14, wherein the indicator is selected from dye classifications selected from the group consisting of azo, indigoid, xanthene, anthraquinone, fluoran, quinoline and combinations thereof.

27. A system for disinfecting a surface comprising:
means for contacting a surface to be disinfected with ozone, wherein one or more substrates, impregnated with an indicator to establish a first color, are proximate to the surface and wherein the ozone gradually oxidizes the indicator to a second color or shade; and
means for comparing the second color or shade to known colors or shades of the indicator that correspond to different ozone Ct values.

28. The system of claim 27, wherein the ozone is dissolved in water or in an organic solvent.

29. The system of claim 27, wherein the ozone is gaseous.

30. The system of claim 27, wherein attaining a target Ct value provides a predetermined level of disinfection.

31. The system of claim 27, wherein the surface is selected from the group consisting of beef, pork, poultry, fish, fruits, vegetables, and combinations thereof.

32. The system of claim 27 wherein the surface is the surface of food.

33. The system of claim 27, wherein the means for contacting the surface is selected from the group consisting of floating, spraying, dunking, immersing, mixing, washing and combinations thereof.

34. The system of claim 27, wherein the target Ct value is between about 1 ppm minute and about 300 ppm minutes.

35. The system of claim 27, wherein the substrates are positioned proximate to the surface by a process selected from free-floating the substrate, attaching the substrate to the surface, attaching the substrate to a holder or combinations thereof.

36. The system of claim 27, wherein the one or more substrates is selected from the group consisting of nylon, polypropylene, vinyl, polytetrafluoroethylene, latex, cellulose, glass and combinations thereof.

37. The system of claim 27, wherein the one or more substrates is any porous material with a rough surface having an affinity for dyeing.

38. The system of claim 27, wherein the one or more substrates is selected from paper, plastic or combinations thereof.

39. The system of claim 27, wherein the indicator is selected from dye classifications selected from the group consisting of azo, indigoid, xanthene, anthraquinone, fluoran, quinoline and combinations thereof.
